(19) United States
(12) Patent Application Publication
(10) Pub. No.: US 2004/0098203 A1
(43) Pub. Date: May 20, 2004

(54) Parallel biological sequence alignments

(76) Inventor: Torbjorn Rognes, Oslo (NO)

Correspondence Address: HARNESS, DICKEY & PIERCE, P.L.C., P.O. BOX 828, BLOOMFIELD HILLS, MI, 48303, US

(21) Appl. No.: 10/471,897

(22) PCT Filed: Jan. 31, 2002
(86) PCT No.: PCT/NO02/00041

(30) Foreign Application Priority Data
Mar. 16, 2001 (NO) 20011362

(51) Int. Cl. G06F 19/00; G01N 33/48; G01N 33/50

(52) U.S. Cl. 702/20

(57) ABSTRACT

A fast and sensitive new biological sequence alignment and database search method has been invented specifically to exploit the advantages of the SIMD technology high sensitivity was reached by a combination of two main factors. First, the computation of the exact optimal un-gapped alignment score of each diagonal in the alignment was performed. Secondly, a novel heuristic for estimating a gapped alignment score taking into account the amount of sequence similarity on several diagonals in the alignment matrix was employed. This estimate is used to identify a 1% fraction of the most interesting database sequences that are subsequently aligned with the query sequence by the Smith-Waterman method. The rapidity of the method was achieved using a very efficient computation of the ungapped alignment scores of all diagonals. The heuristic for computing the estimated gapped alignment score is also fast.

### FIELD OF THE INVENTION

The present invention relates to the field of biological database searches and more specifically, the invention relates to a method, a computer readable device comprising instructions and an electronic device comprising circuitry for performing searches in genetic sequence databases in order to identify the database sequences most closely related to a given query sequence in accordance with the independent claims 1,3,5,7,10 and 12 and variations of the inventions thereof in the dependent claims 2,4,6,8,9 and 13.

### BACKGROUND OF THE INVENTION

The total size of the public sequence databases is rapidly increasing and has doubled in the last 6 months. Searching databases for sequences similar to a given sequence is one of the most fundamental and important tools for predicting structural and functional properties of uncharacterised proteins. The availability of good tools for performing these searches is hence important. There exist a number of tools with varying speed and sensitivity. The Smith-Waterman algorithm as known to a person skilled in the art is generally considered to be the most sensitive, but long computation times limit the use of this algorithm. To increase speed, several heuristic alternatives has been developed, such as FASTA ((Pearson and Lipman, 1988) BLAST (Altschul et al., 1990, Altschul et al., 1997), and WU-BLAST (W. Gish, http://blast.wust1.edu). These programs sacrifice sensitivity for speed, and therefore more distant sequence relationships may escape detection. Special-purpose hardware with parallel processing capabilities has also been constructed to perform Smith-Waterman searches at high speed, but these machines are quite expensive.

A form of parallel processing capability termed Single-Instruction Multiple-Data (SIMD) technology enables microprocessors to perform the same operation (logical, arithmetic or other) in parallel on several independent data sources. It is possible to exploit this by dividing wide registers into smaller units in the form of micro-parallelism or SIMD Within A Register (SWAR). However, modern microprocessors have added special registers and instructions to make the SIMD technology easier to use. The technology is included in some of the most widely used modern microprocessors, including the Intel Pentium MMX, II and III. A form of SIMD technology called MMX (MultiMedia eXtensions) is included in the former two, while an enhanced version called SSE (Streaming SIMD Extensions) is included in the latter. Processing of sound, images and video are the main application areas, but the technology is also useful for other signals, and can be applied to processing of genetic sequences. The present known multimedia technologies utilising these computer architecture principles are known as SIMD, MMX, SSE, SSE2, MAX, MAX-2, MDMD, VIS, AltiVec.

Several investigators have used SIMD technology to speed up the Smith-Waterman algorithm, but there seems to be no heuristic sequence alignment algorithms designed specifically to take advantage of the SIMD technology.

Here, a semi-heuristic method specifically designed to exploit the advantages of the SIMD technology for performing both rapid and sensitive sequence database searches is presented. It has been implemented using the Intel MMX/SSE technology, but can be adapted to other microprocessor architectures.

### DISCLOSURE OF THE INVENTION

For each sequence in the database, similarity search programs compute an alignment score that represent the degree of similarity between the query and the database sequence. This score is based on a substitution score matrix representing the similarity between two symbols, and an affined gap penalty function based on a gap open and a gap extension penalty. The optimal local alignment score can be computed by the dynamic programming algorithm due to Smith and Waterman and Gotoh as known to a person skilled in the art. A path through an m time's n alignment matrix spanned by the query sequence and the database sequence can represent an optimal local alignment. The path consists of patches parallel to the major diagonal, representing matching symbols, and vertical or horizontal patches, representing gaps in the query or database sequence. The score of an alignment is the sum of the substitution scores in the included cells minus the penalty for the gaps. The optimal alignment is the alignment giving the highest score. Usually the path of an optimal alignment consists of a few long diagonal patches connected by short gaps.

Ungapped alignment can be considered as a special case of alignment with infnitely large gap penalties. The interdependence between the diagonals then disappears, and an alignment score can be computed separately for each diagonal and the highest of these is the optimal ungapped alignment score. The alignment score for each diagonal is equal to the maximum partial sum of substitution scores along a continuous part of the diagonal. Many heuristic search algorithms, including FASTA and BLAST, are based on first identifying high-scoring ungapped alignments. In the BioSCAN system (Singh, R. K., Hoffman, D. L., Tell, S. G. and White C. T., 1996) computations of diagonal scores as described above was performed by special purpose hardware as the basis of a database search method.

The sequence alignment performed by most heuristic database search algorithms can be divided into two phases. In the first phase, regions of similarity between the two sequences are identified without considering gaps. In the second phase, gapped alignments are constructed on the basis of the most interesting regions initially identified. The way the initial screening of the database sequences is performed by the traditional heuristic algorithms like FASTA and BLAST leads to increased speed, but also to reduce the sensitivity compared to the Smith-Waterman algorithm. In order for the sequence similarities to be detected by these programs, it is required that the similarity between the sequences is in some way concentrated. Enough similarity must be present within a small group of identical or highly conserved consecutive amino acids on a single diagonal in the alignment matrix. If the similarity between the two sequences is more spread along one diagonal in the alignment matrix, or divided on several diagonals, it may not be detected. The present invention has been designed to avoid these problems, without using the extensive computation time required by the Smith-Waterman algorithm.

### BRIEF DESCRIPTIONS OF THE DRAWINGS

FIG. 1. illustrates that the computation of the diagonal scores is performed efficiently in the order indicated using bands that are 32 diagonals wide using SIMD technology.

FIG. 2. illiustrates computation of the estimated gapped alignment score where the numbers within the matrix are the temporary scores, eᵢ, along the diagonals from the initial computation of the diagonal scores. The numbers right outside of the matrix are the optimal ungapped alignment scores, S_{d}, for each diagonal. The second numbers outside the matrix are the temporary scores, u_{d}, used in the calculation of the estimated gapped alignment score, T, which in this example is 3+11+1+22=37. The BLOSUM62 matrix was used in combination with the parameters q=11, r=11 and c=3 in this example. The calculations are performed in order of increasing diagonal numbers.

FIG. 3a and FIG. 3b illustrates comparison of database search sensitivity and selectivity, and the sensitivity (coverage) versus the selectivity (EPQ, errors per query) is plotted for a range of database search programs. Using either (a) the BLOSUM50 matrix and a 10+2 k gap penalty, or (b) the BLOSUM62 matrix and an 11+k gap penalty.

FIG. 4 illustrates comparison of database search speed where the search time versus the query sequence length is plotted for the different search programs and the 11 query sequences (see results). The search time used is the total CPU time of the fastest of 3 consecutive runs on a minimally loaded computer. With a database of only 29 MB and 128 MB of RAM, the entire database was cached in the computer's RAM; disk-reading time should then be negligible.

### PREFERRED EMBODIMENT OF THE INVENTION

The preferred embodiment will be described with reference to the drawings. In the first phase, both FASTA and BLAST scan for short regions of 1-3 consecutive amino acids that are identical or very similar between the two sequences. More precisely, FASTA requires 1 or 2 amino acids (specified by the ktup parameter) to be identical. By default, BLAST requires 3 amino acids to be very similar, that is, the sum of the three score matrix elements corresponding to the three pairs of amino acids must exceed a certain limit. NCBI BLAST version 2 additionally requires that there are two such groups on the same diagonal within a distance of about 40 amino acids. Hence, if the similarity is more distributed along one diagonal it may be undetected by the present heuristic methods. This weakness is overcome in the present invention by calculating the exact ungapped alignment score for each diagonal. Consider a query sequence A of length m, a database sequence B of length n, and a score matrix Z. The optimal ungapped alignment score S_{d} for diagonal d, which is a maximum partial sum of substitution scores along the diagonal, can be computed iteratively using equations 1-3 below with row i going from g to h, where g=max(1,1−d) and h=min(m,n−d):

eᵢ=max{eᵢ₋₁+Z[Aᵢ, B_{i+d}], 0}e_{g-1}=0 (1)

fᵢ=max{fᵢ₋₁, eᵢ}f_{g-1}=0 (2)

S_{d}=fₕ (3)

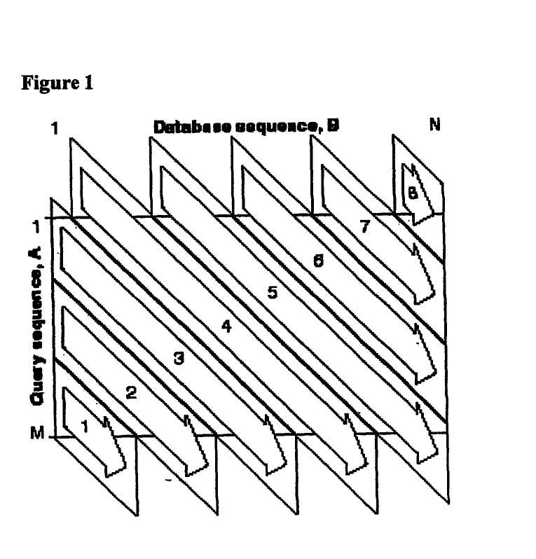

An exceptionally fast way to perform this operation using the SIMD technology has been found. In the present implementation, an 8-way parallel processing approach is used. The calculation of eᵢ and fᵢ is performed in diagonal bands, 32 cells wide, parallel to the major diagonal in the matrix. The computation is started in the lower left corner of the matrix, and continued in the order indicated in FIG. 1. This arrangement enables a very efficient computation. The 64-bit MMX registers were divided into eight 8-bit units in order to get the largest number of parallel computations. Four of the MMX registers are used for the eᵢ values, while the remaining four registers are used for the fᵢ values. The resulting maximum partial sum of score on each diagonal is saved and used in the subsequent calculations.

When computing eᵢ by adding the substitution scores from Z to eᵢ₋₁, the signed and saturated addition instruction PADDSB (packed add with saturation, byte) is used. When fᵢ is computed as the maximum of fᵢ₋₁ and eᵢ, the unsigned maximum instruction PMAXUB (packed unsigned integer byte maximum) is used. Using only a byte for eᵢ and fᵢ limits the precision of the calculations to 8 bits. However, in order to enable the use of the PADDSB and PMAXUB instructions, the range had to be further limited to 0-127 and represented by values in the range −128 to −1 by biasing eᵢ and fᵢ by −128. Limiting the precision to 7 bits poses no real problems because a score close to 127 will always be considered significant and the correct score will be computed in the subsequent computations of a more accurate alignment score with a wider score range as described below.

Initially, a query sequence profile is created for the query sequence in order to speed up computations. This profile contains the substitution score for matching each of the possible amino acid symbols with each symbol in the query sequence. The scores for matching symbol A with each symbol in the query sequence is followed by the scores for matching symbol B with each symbol in the query sequence, and so on. The scores for matching one database symbol with a group of 32 consecutive symbols from the query sequence could then be retrieved by loading four MMX registers from consecutive addresses. Usually, the entire query profile will be kept in the first level cache of the CPU, resulting in very fast accesses.

Computation of an approximate gapped alignment score

The second phase of sequence alignment also has its problems. Sequence similarities may pass undetected if the similarity is evenly distributed on several diagonals, in which case the optimal alignment contains many gaps. Many heuristic algorithms select a small fraction (e.g. 2%) of the database sequences for a more rigorous examination using an optimal alignment algorithm within a band (FASTA) or region (NCBI BLAST version 2) surrounding the most interesting initially identified regions. NCBI BLAST uses only the score of the highest-scoring initial region to determine whether a more accurate gapped alignment should be constructed. It is hence unable to recognise an otherwise significant alignment if none of the high-scoring segment pairs (HSPs) found has a score above about 40 with a typical-length query. FASTA is more advanced, and makes this decision based on an approximate gapped alignment score, initn, computed by joining several compatible regions and subtracting joining penalties from their scores.

The present invention employs a new heuristic for computing an estimated gapped alignment score, which is used to select the most interesting fraction of database sequences for further examination. When the ungapped alignment score for all diagonals have been found, as described previously, the scores for each diagonal are combined in a new inter-diagonal scoring function, which is also a kind of maximum partial sum of scores function. Using this function, high-scoring regions on neighbouring diagonals will receive a high score. The chosen inter-diagonal scoring function has been found to be very effective in filtering the sequence database. Some simplifications are performed in this calculation of an approximate gapped alignment score. First, only the single highest-scoring region on each diagonal is considered. In addition, only diagonals scoring above average are considered interesting. This is achieved by deducting the expected score, c, for a diagonal from the score of each diagonal. This expected score is dependent on the length of the query sequence. Equation 4 below is used to estimate the expected diagonal score:

$$c = \frac{ln K {m}^{′}}{λ} \qquad ( 4 )$$

Here, K and λ are the parameters for calculating the statistical significance of ungapped alignment as described by Karlin and Altschul (1990). The values estimated by Altschul and Gish (1996) for the given substitution score matrix was used. The edge-corrected length of the query sequence is represented by m′. The relative position of regions on different diagonals is not considered, only the distance between the diagonals, which are used for computing gap penalties. The resulting scores are added up for neighbouring diagonals, and the optimal combination of these is found. The approximate gapped alignment score T for an alignment is computed by equations 5-7 below, with diagonal d going from 1−m to n−1:

u_{d}=max{u_{d-1}+max[S_{d}−c−q, 0]−r, 0}u₋ₘ=0 (5)

v_{d}=max{v_{d-1}, u_{d}}v₋ₘ=0 (6)

T=c+q+r+vₙ₋₁ (7)

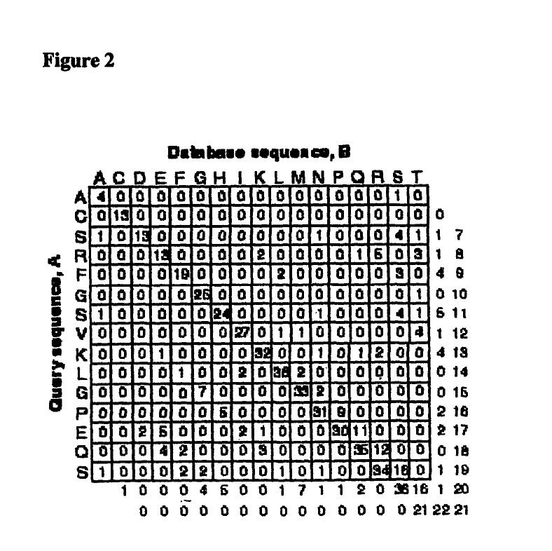

Here, q and r are the gap open and extension penalties, respectively. A calculation example is shown in FIG. 2. The use of this approximate gapped alignment scoring function allows the identification of sequence similarities that are distributed so evenly on several diagonals that an alignment with gaps is necessary to detect the similarity. The fraction containing about 1% of the highest scoring database sequences is finally subject to a rigorous Smith-Waterman alignment that is also implemented using SIMD technology as depicted in a parallel patent application attorney document E21584. The cut-off value, w, for selecting the about 1% (assuming random sequences) of the highest-scoring sequences is calculated by equation 8 below:

$$w = \frac{ln ( K {m}^{′} {n}^{′} ) + ln ( 100 )}{λ} \qquad ( 8 )$$

Here, K and λ are the parameters for calculating the statistical significance of gapped alignment as described and estimated by Altschul and Gish (1996). The edge effect corrected lengths of the query and database sequences are represented by m′ and n′. This formula for the cut-off value assumes a score distribution that is equal to an optimal gapped alignment score. This approximation has been shown in practice to give a useful cut-off value.

Results

The ability of the present invention to detect homologous proteins was evaluated and compared to other methods for pair wise sequence alignment. The programs evaluated are listed in table 1.

TABLE 1
Database search programs tested

| Program | Version | Description | Reference |
|---|---|---|---|
| SSEARCH | 3.3t08 | Full dynamic-programming | Pearson, W. R. (1991) |
| FASTA | 3.3t08 | Heuristic, tested with both ktup 1 and 2 | Pearson, W. R. and Lipma |
| | | | (1988) |
| NCBI BLAST | 2.0.14 | Heuristic | Altschul, S. F., Madden, T. |
| | | | Schaffer, A. A., Zhang, J., |
| | | | Zhang, Z., Miller, W. and |
| | | | Lipman, D. J. (1997) |
| NCBI BLAST | 1.4.9 | Heuristic, ungapped alignments | Altschul, S. F., Gish, W., |
| | | | Miller, W., Myers, E. W. a |
| | | | Lipman, D. J. (1990) |
| WU-BLAST | 2.0a19 | Heuristic | W. Gish http://blast.wust |
| Present | 1.9.7 | Heuristic, SIMD implementation | Present invention |
| invention | | | |
| SWMMX | 1.9.7 | Full dynamic-programming, | parallel patent application |
| | | SIMD implementation | attorney document E2158 |
| SALSA | 1.8.4 | Heuristic | Brenner, S. E., Chothia, C. |
| | | | Hubbard, T. J. (1998) |

The performance assessment was carried out as described by Brenner et al. (1998). The evaluation is based on the PDB40D-B database (provided by Brenner et al.), which consists of domains of proteins from the SCOP database (Murzin, A. G., Brenner, S. E., Hubbard, T. and Chothia C., 1995) classified according to their tertiary structure. The ability of the different database search methods to correctly identify the “true” homologous proteins in this database can be tested using the SCOP super-family classification as a reference. The PDB40D-B database contains 1323 protein domains that are less than 40% identical to each other. There are 9044 ordered pairs of domains belonging to the same super-family out of a total of 1 749 006 ordered pairs. Both the coverage (true positives), defined as the fraction of homologous pairs correctly identified, and the number of errors per query (EPQ) (false positives), defined as the number of incorrectly reported sequences per query sequence, was examined. Thus both the ability of a program to detect homologous protein pairs (sensitivity) and its ability to discriminate between the homologous and non-homologous pairs (selectivity) is assessed. An all-versus-all comparison of the sequences in the database was performed. All hits were recorded and sorted on the statistical significance ranking parameter reported by the programs, e.g. the P-value (match probability) for NCBI BLAST version 1 and WU-BLAST, and the E-value (expected matches) for the other programs. The number of correctly and incorrectly identified homologous pairs was counted and recorded for each reported significance level.

The database scanning approach, the choice of score matrix, the choice of gap penalty scheme, and the method of statistical evaluation of the scores are four different aspects of a database search that may be assessed individually. Regarding the first aspect, the SSEARCH and SWMMX programs both take a full dynamic programming approach to database scanning, while the other programs use different heuristics.

By default, the SSEARCH and FASTA programs use the BLOSUM50 amino acid substitution score matrix (Henikoff; S. and Henikoff, J. G., 1992) in combination with a gap penalty of 10+2 k. On the other hand, NCBI BLAST version 2, SALSA, the present invention and SWMMX use the BLOSUM62 matrix with gap penalty of 11+k by default. To make a fair comparison, both scoring schemes were tested. However, the former set of matrix and gap penalty parameters were not tested with SALSA and NCBI BLAST version 2 because these programs would not run with these parameters due to the lack of corresponding precomputed Karlin-Altschul constants.

There are different ways of calculating the statistical significance of a match based on its raw alignment score. The SSEARCH and FASTA programs by default calculate the statistical significance of the matches by regression against the length of the library sequence. However, by specifying the “−z 3” option, these programs will use Karlin-Altschul statistics with precomputed Lambda, K and H constants. NCBI BLAST VERSION 1 by default uses sum-statistics (Karlin S. and Altschul S.F., 1993) while the other programs use Karlin-Altschul statistics (Karlin, S. and Altschul, S. F., 1990). There are some differences between the implementations of the Karlin-Altschul statistics, related to the method of length-correction, and in the K, Lambda and H constants. For the FASTA and SSEARCH programs both the default and the Karlin-Altschul method of statistical significance calculation were examined.

All programs were run with options set to show up to 500 scores with expect value below 10 and no alignments. WU-BLAST and SALSA were run the recommended “postsw” and “−o” options, respectively, in order to compute the optimal alignment score for all reported hits.

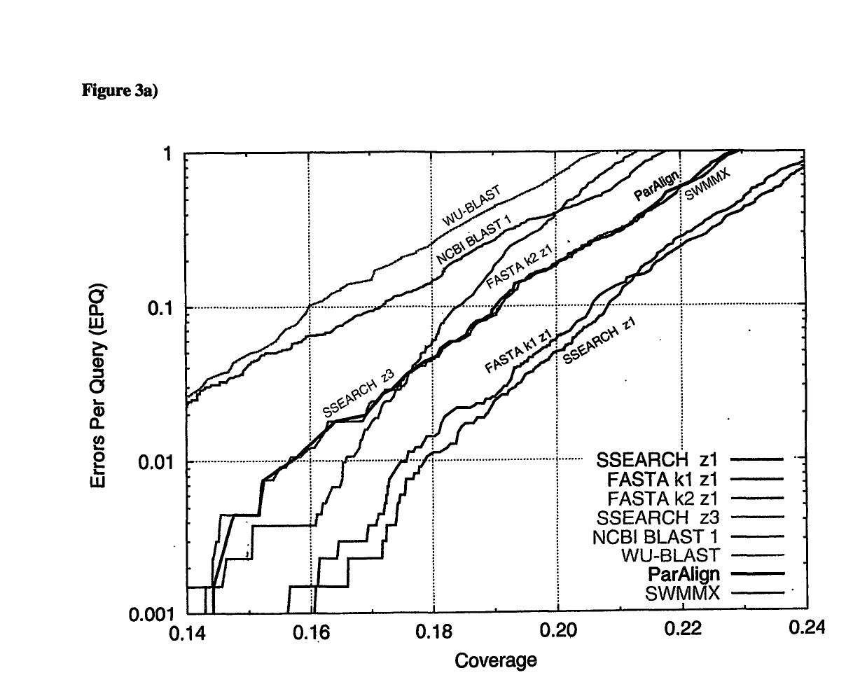

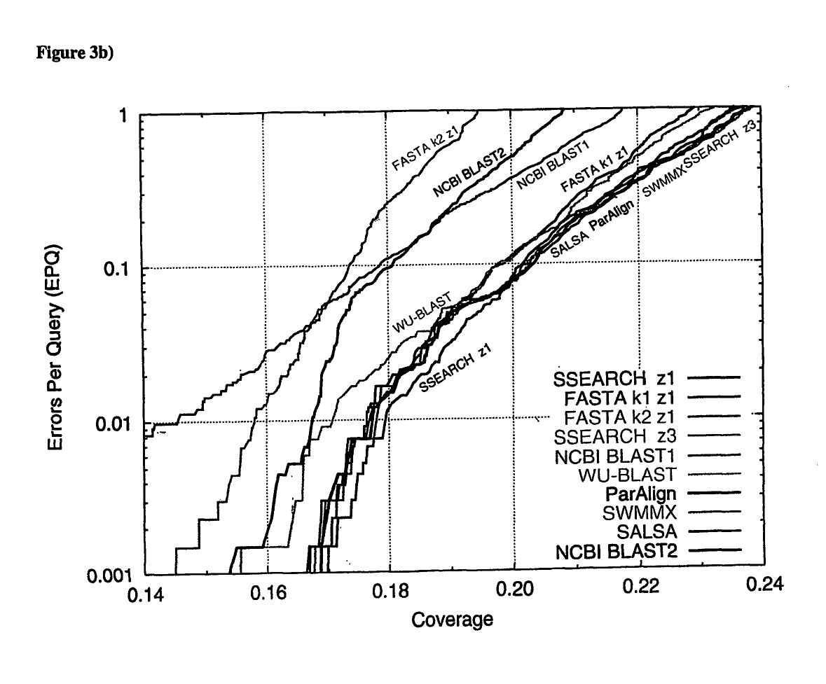

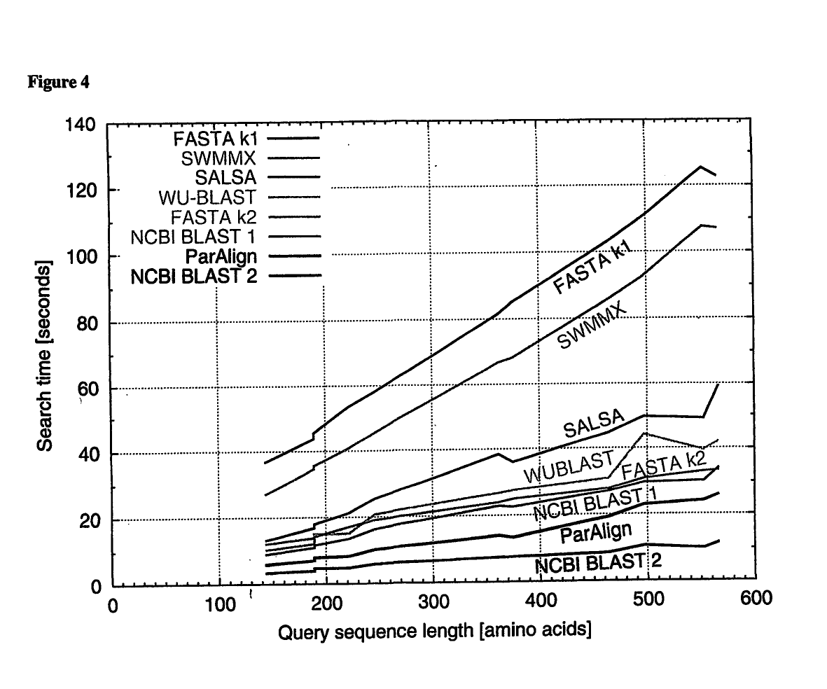

Plots of the performance are shown in FIG. 3. The SSEARCH program using default (length-regression) statistics achieved the overall best performance. When Karlin-Altschul statistics were employed, the best results were obtained with the BLOSUM62 matrix and 11+k gap penalties. In that case, the best performance was obtained with the present invention, SSEARCH, SWMMX and SALSA, which all performed essentially equally. FASTA with ktup 1 and WU-BLAST performed nearly as good, but PASTA with ktup 2 and both versions of NCBI BLAST performed significantly worse. Because of the rather small size of the PDB40D-B database compared to the sizes of databases usually searched, the speed of the various algorithms was assessed by searching the SWISS-PROT release 39 database (Bairoch, A. and Apweiler, R., 2000). A set of 11 test query sequences (SWISS-PROT accession numbers P00762, P01008, P01111, P02232, P03435, P05013, P07327, P10318, P10635, P14942, P25705) with lengths in the range 143 to 567 was used. These test sequences have previously been used in the evaluation of NCBI BLAST version 2. The BLOSUM62 matrix was used in combination with a gap penalty of 11+k; other program options were set as described earlier. A plot of the search time versus the query sequence length for each query sequence and program is shown in FIG. 4. Overall, the present invention was found to be 4.4 times faster than the Smith-Waterman algorithm as implemented in SWMMX and 28 times faster than the implementation in SSEARCH. The present invention was also found to be faster than any of the heuristic algorithms except for NCBI BLAST version 2, which was twice as fast as the present invention. The present invention was 1.8 times faster than WU-BLAST, 2.3 times faster than SALSA, 5.4 times faster than FASTA with ktup 1, and 1.6 times faster than FASTA with ktup 2. There was no noticeable speed difference of the two different statistical methods for FASTA and SSEARCH.

The speed of the present invention was only surpassed by NCBI BLAST version 2, which proved significantly less sensitive in this test. Considerably improved speed will probably be obtained with the present invention when 128-bit SIMD technology becomes available. Most other heuristic alignment algorithms cannot take advantage of improvements in SIMD technology.

Modern methods that take advantage of the information gained from multiple related protein matches in the database are often able to detect many more homologues than the simple pair wise alignment methods. Such programs include PSI-BLAST, Sam-T98 and MISS. However all these methods initially depends on a pair wise alignment method. Increased speed and/or sensitivity of the overall iterative method can probably be obtained by using an improved pair wise alignment method, of which the present invention might be a good choice.

It is likely that the present invention can be implemented efficiently also on most other microprocessors with SIMD technology. Future computer generations will probably included more advanced SIMD technology, e.g. microprocessors with 16-way parallel processing, that might allow even faster performance. Equivalents to the above implementations can be other computer systems using digital signal processing performing the disclosed method. Dedicated hardware solutions in the form of electronic circuits that incorporates the described method, e.g. an application specific integrated circuit (ASIC), field programmable gate arrays (FPGA), or custom very large scale integration (VLSI) chips. Using VHDL hardware description language of the method outlined in this disclosure of the present invention, any form of electronic device implementation of the method can be achieved as long as the VHDL synthesize tool support the chosen technology as well known to a person skilled in the art.

1. Method for performing searches in biological sequence databases in order to identify the database sequences most closely related to a given query sequence where searches are carried out by comparing the query sequence to each database sequence and by computing Smith-Waterman similarity scores representing the amount of similarity between a query sequence A of length M and a database sequence B of length N and a symbol substitution score value matrix Z and an open penalty gap q and a gap extension penalty r where the method comprises the steps of:
calculating an approximate similarity score value using a highest local alignment score between said query sequence A and said database sequence B without considering the open penalty gap q and the gap extension penalty r; and
comparing said approximate similarity score value with a predefined threshold value rejecting said database sequence B as non-similar with said query sequence if the value of said similarity score is below or above said threshold value depending on the signs and absolute values of said threshold value and said approximate similarity score value; and
calculating a precise Smith-Waterman similarity score value of said query sequence A and said database sequence B if said sequence B is not rejected when tested against said threshold value resulting in generating a partial list of database sequences and their accurate scores.

2. Method according to claim 1 further comprising the step of:
calculating said local alignment score value between said query sequence A of length M and said database sequence B of length N by combining all the M+N−1 alignment combinations without considering the open penalty gap q and the gap extension penalty r using said symbol substitution matrix Z containing score values for said query sequence A and said database sequence B using the local maximum score for each alignment as the maximum partial sum of substitution scores from Z representing a continuous sequence of symbol pairs from the alignment of said sequence A and said sequence B.

3. Method for performing searches in biological sequence databases in order to identify the database sequences most closely related to a given query sequence where searches are carried out by comparing the query sequence to each database sequence and by computing Smith-Waterman similarity scores representing the amount of similarity between a query sequence A of length M and a database sequence B of length N and a symbol substitution score value matrix Z and an open penalty gap q and a gap extension penalty r where the method comprises the steps of:
calculating an approximate similarity score value using a highest local alignment score between said query sequence A and said database sequence B without considering the open penalty gap q and the gap extension penalty r; and
comparing said approximate similarity score value with a predefined threshold value rejecting said database sequence B as non-similar with said query sequence if the value of said similarity score is below or above said threshold value depending on the signs and absolute values of said threshold value and said approximate similarity score value; and
calculating a precise Smith-Waterman similarity score value of said query sequence A and said database sequence B if said sequence B is not rejected when tested against said threshold value resulting in generating a new filtered database of biological sequences.

4. Method according to claim 3 further comprising the step of:
calculating said local alignment score value between said query sequence A of length M and said database sequence B of length N by combining all the M+N−1 alignment combinations without considering the open penalty gap q and the gap extension penalty r using said symbol substitution matrix Z containing score values for said query sequence A and said database sequence B using the local maximum score for each alignment as the maximum partial sum of substitution scores from Z representing a continuous sequence of symbol pairs from the alignment of said sequence A and said sequence B.

5. Computer readable device containing computer instructions that provide searches in biological sequence databases in order to identify the database sequences most closely related to a given query sequence where searches are carried out by comparing the query sequence to each database sequence and by computing Smith-Waterman similarity scores representing the amount of similarity between a query sequence A of length M and a database sequence B of length N and a symbol substitution score value matrix Z and an open penalty gap q and a gap extension penalty r comprising the instructions of:
calculating an approximate similarity score value using a highest local alignment score between said query sequence A and said database sequence B without considering the open penalty gap q and the gap extension penalty r; and
comparing said approximate similarity score value with a predefined threshold value rejecting said database sequence B as non-similar with said query sequence if the value of said similarity score is below or above said threshold value depending on the signs and absolute values of said threshold value and said approximate similarity score value; and
calculating a precise Smith-Waterman similarity score value of said query sequence A and said database sequence B if said sequence B is not rejected when tested against said threshold value resulting in generating a partial list of database sequences and their accurate scores.

6. Computer readable device according to claim 5 further comprising the instructions of:
calculating said local alignment score value between said query sequence A of length M and said database sequence B of length N by combining all the M+N−1 alignment combinations without considering the open penalty gap q and the gap extension penalty r using said symbol substitution matrix Z containing score values for said query sequence A and said database sequence B using the local maximum score for each alignment as the maximum partial sum of substitution scores from Z representing a continuous sequence of symbol pairs from the alignment of said sequence A and said sequence B.

7. Computer readable device containing computer instructions that provide searches in biological sequence databases in order to identify the database sequences most closely related to a given query sequence where searches are carried out by comparing the query sequence to each database sequence and by computing Smith-Waterman similarity scores representing the amount of similarity between a query sequence A of length M and a database sequence B of length N and a symbol substitution score value matrix Z and an open penalty gap q and a gap extension penalty r comprising the instructions of:
calculating an approximate similarity score value using a highest local alignment score between said query sequence A and said database sequence B without considering the open penalty gap q and the gap extension penalty r; and
comparing said approximate similarity score value with a predefined threshold value rejecting said database sequence B as non-similar with said query sequence if the value of said similarity score is below or above said threshold value depending on the signs and absolute values of said threshold value and said approximate similarity score value; and
calculating a precise Smith-Waterman similarity score value of said query sequence A and said database sequence B if said sequence B is not rejected when tested against said threshold value resulting in generating a new filtered database of biological sequences.

8. Computer readable device according to claim 7 further comprising the instructions of:
calculating said local alignment score value between said query sequence A of length M and said database sequence B of length N by combining all the M+N−1 alignment combinations without considering the open penalty gap q and the gap extension penalty r using said symbol substitution matrix Z containing score values for said query sequence A and said database sequence B using the local maximum score for each alignment as the maximum partial sum of substitution scores from Z representing a continuous sequence of symbol pairs from the alignment of said sequence A and said sequence B.

9. Computer readable device according to one of the claims 5,6,7 or 8 comprising instructions utilizing the parallel computing possibilities of the multimedia technology type instructions known as SIMD, MMX, SSE, SSE2, MAX, MAX-2, MDMD, VIS, AltiVec or related technologies.

10. Electronic device of type ASIC (Application Specific Integrated Circuit) or VLSI (Full Custom Integrated Circuit) or FPGA (Field programmable Gate Array) or any other type of programmable electronic device with electronic circuitry performing searches in biological sequence databases in order to identify the database sequences most closely related to a given query sequence where searches are carried out by comparing the query sequence to each database sequence and by computing Smith-Waterman similarity scores representing the amount of similarity between a query sequence A of length M and a database sequence B of length N and a symbol substitution score value matrix Z and an open penalty gap q and a gap extension penalty r where said device comprises circuitry for:
calculating an approximate similarity score value using a highest local alignment score between said query sequence A and said database sequence B without considering the open penalty gap q and the gap extension penalty r; and
comparing said approximate similarity score value with a predefined threshold value rejecting said database sequence B as non-similar with said query sequence if the value of said similarity score is below or above said threshold value depending on the signs and absolute values of said threshold value and said approximate similarity score value; and
calculating a precise Smith-Waterman similarity score value for said query sequence A and said database sequence B if said sequence B is not rejected when tested against said threshold value resulting in generating a partial list of database sequences and their accurate scores.

11. Electronic device according to claim 10 further comprising circuitry for:
calculating said local alignment score value between said query sequence A of length M and said database sequence B of length N by combining all the M+N−1 alignment combinations without considering the open penalty gap q and the gap extension penalty r using said symbol substitution matrix Z containing score values for the said query sequence A and the said database sequence B using the local maximum score for each alignment as the maximum partial sum of substitution scores from Z representing a continuous sequence of symbol pairs from the alignment of said sequence A and said sequence B.

12. Electronic device of type ASIC (Application Specific Integrated Circuit) or VLSI (Full Custom Integrated Circuit) or FPGA (Field programmable Gate Array) or any other type of programmable electronic device with electronic circuitry performing searches in biological sequence databases in order to identify the database sequences most closely related to a given query sequence where searches are carried out by comparing the query sequence to each database sequence and by computing Smith-Waterman similarity scores representing the amount of similarity between a query sequence A of length M and a database sequence B of length N and a symbol substitution score value matrix Z and an open penalty gap q and a gap extension penalty r where said device comprises circuitry for:
calculating an approximate similarity score value using a highest local alignment score between said query sequence A and said database sequence B without considering the open penalty gap q and the gap extension penalty r; and
comparing said approximate similarity score value with a predefined threshold value rejecting said database sequence B as non-similar with said query sequence if the value of said similarity score is below or above said threshold value depending on the signs and absolute values of said threshold value and said approximate similarity score value; and
calculating a precise Smith-Waterman similarity score value of said query sequence A and said database sequence B if said sequence B is not rejected when tested against said threshold value resulting in generating a new filtered database of biological sequences.

13. Electronic device according to claim 12 further comprising circuitry for:
calculating said local alignment score value between said query sequence A of length M and said database sequence B of length N by combining all the M+N−1 alignment combinations without considering the open penalty gap q and the gap extension penalty r using said symbol substitution matrix Z containing score values for the said query sequence A and the said database sequence B using the local maximum score for each alignment as the maximum partial sum of substitution scores from Z representing a continuous sequence of symbol pairs from the alignment of said sequence A and said sequence B.
